(19) United States
(12) Patent Application Publication
(10) Pub. No.: US 2019/0029559 A1
(43) Pub. Date: Jan. 31, 2019

(54) METHOD FOR A DIRECT POSITIONING OF A REGION OF INTEREST OF A PATIENT INSIDE A SCANNER OF A MAGNETIC RESONANCE IMAGING APPARATUS

(71) Applicant: Siemens Healthcare GmbH, Erlangen (DE)

(72) Inventors: Stephan Nufer, Erlangen (DE); Dominik Paul, Bubenreuth (DE); George William Ferguson, Erlangen (DE)

(73) Assignee: Siemens Healthcare GmbH, Erlangen (DE)

(21) Appl. No.: 16/042,385

(22) Filed: Jul. 23, 2018

(30) Foreign Application Priority Data
Jul. 25, 2017 (EP) 17183011

(51) Int. Cl. A61B 5/055; A61B 5/00; G01R 33/34

(52) U.S. Cl. CPC A61B 5/0555; G01R 33/543; G01R 33/34092; A61B 5/0037

(57) ABSTRACT

In a method for a direct positioning of a region of interest of a patient inside a basic field magnet of a canner of a magnetic resonance imaging apparatus, a patient is positioned on a patient table, and at least one local RF-coil is positioned on or close to the patient on a region of interest that is to be examined. A distance between the position of the at least one local RF-coil and the isocenter of the basic field magnet is determined. The local RF-coil together with the patient table is moved automatically along the z-direction for at least the determined distance, so that a center of the local RF-coil is in or at least at the z-position of the isocenter.

### BACKGROUND OF THE INVENTION

### Field of the Invention

The present invention relates to a method for a direct positioning of a region of interest of a patient inside a scanner of a magnetic resonance imaging apparatus. Thus, the present invention relates to the technical field of performing an examination of a patient with a magnetic resonance imaging apparatus.

### Description of the Prior Art

In a magnet resonance (MR) imaging examination, the patient is placed on a patient table and a local RF-coil, such as a local receiver coil, is placed near or on top of a region of interest of the patient, from which MR data are to be acquired. A laser pointer is usually used to designate the region of interest. The region of interest will then be moved into the isocenter of the scanner of the magnetic resonance imaging apparatus in order to achieve the best imaging quality. After this step of setting up the patient, several localizer images are usually acquired, in particular in all three spatial directions, in order to obtain an overview of the patient's anatomy and in order to plan the level of detail needed to perform the clinical imaging. If the region of interest is not located in the isocenter, a correction of the table position must be performed and new localizer images need to be acquired. Such a repeated acquisition of localizer images takes additional time, and nearly all manual steps needed for repositioning also require additional time. Thus, the time for adjustments usually takes too much time. This in turn makes the workflow for preparing a patient for an examination with a magnetic resonance imaging apparatus time-consuming, so that the utilization rate of a magnetic resonance imaging apparatus is decreased.

An exact positioning of a local RF coil relative to inner organs or to bones, such as a hip or the like, is especially challenging. Thus, repeated acquisitions are often needed because while scanning the hip, for example, it is hard to position the local RF-coil exactly on the femoral head. This means that after a first localizer acquisition, repositioning of the RF-coil as described above or repositioning of the graphical slice planning objects is often needed.

### SUMMARY OF THE INVENTION

Therefore, an object of the present invention is to provide a method for shortening the time needed for the preparation step and therewith for improving a utilization rate of a magnetic resonance imaging apparatus and to provide a magnetic resonance imaging apparatus that can be used effectively.

According to the invention, a method for a direct positioning of a region of interest of a patient inside a basic field magnet of a magnetic resonance imaging apparatus is provided that has steps.

The patient is positioned on a patient table, and at least one local RF-coil is positioned on or close to the patient on a region of interest that is to be examined. A distance between the position of the at least one local RF-coil and the isocenter of the scanner is determined in a control computer of the magnetic resonance imaging apparatus. The local RF-coil, together with the patient table, moved automatically by the control computer along a z-direction in the scanner for at least the determined distance, so that the center of the local RF-coil is in or at least at z-position of the isocenter.

According to the inventive method, the patient is placed on a patient table that is designed to be movable along the z-direction of the scanner, in particular along the body length of the patient, from an exterior of the scanner into the interior of the scanner. In a next step, at least one local RF-coil for receiving diagnostic (MR) data, is positioned on or close to the patient directly on a region of interest that is to be examined. For example, the local RF-coil may be plugged into a given socket in the scanner, such as a socket on the patient table. In a next step, the distance between the position of the at least one local RF-coil and the isocenter of the scanner is determined, the latter being a fixed known point in the scanner or more precisely in the basic field magnet of the scanner. The determination of this distance can either be calculated directly if the local RF coil is plugged into a given connection, or can be detected by a detection unit and can be evaluated afterwards. After the determination of the distance between the local RF-coil and the isocenter, the local RF-coil together the patient table is moved automatically through the determined distance along the z-direction so that the center of the local RF-coil is in or at least at the z-position of the isocenter. An advantage of the inventive method is that the region of interest can be directly moved into the isocenter of the magnet. There is no need to use a laser pointer anymore. Thus the workflow for preparing the patient can be performed more quickly, and the utilization of the magnetic resonance imaging apparatus can be increased.

In an embodiment of the invention, the position of the at least one local RF-coil is predetermined, or is detected by a detection unit. The position of the at least one local RF-coil is predetermined if the at least one local RF-coil is plugged into a given socket connection, so that the magnetic resonance imaging apparatus detects the RF-coil automatically. Alternatively the RF-coil can be detected by a detection unit, so that the distance between the position of the RF-coil and the isocenter can be calculated. The detection unit has a camera, and/or a sensor inside the RF-coil. If a camera system is used, it is preferably a 2D or a 3D camera, which produces optical images. If a sensor is used, the sensor is preferably located inside the RF-coil and is preferably designed as a Hall sensor. The information about the depth is important in order to know the exact position of the RF-Coil. Such parameters are used for graphical slice planning. The determination and the evaluation of the position of the RF-coil within regard to the isocenter can be performed automatically. This has the advantage that the preparation steps can be performed quickly, in particular, without losing time for adjustment or repositioning work steps.

In a further embodiment of the invention, localizer data are acquired by the magnetic resonance imaging apparatus while the at least one local RF-coil is moved into the isocenter. As the patient table is moved into the isocenter, localizer images can be detected, in particular along the z-direction, and/or the x-direction along which the RF-coil extends. This means the acquisition of the localizer images can be focused along the extent of the RF coil. In particular the localizer images are obtained in the z- and the x-directions along the at least local RF-coil. This means that the localizer images can be acquired with as many slices as are necessary in order to obtain a detailed overview of the region of interest, and can be acquired restricted to the extent of the RF coil. This has the advantage that the localizer images can be acquired with a high image quality while the patient table is being moved into the isocenter, without losing time.

According to a further embodiment of the invention, the localizer data are acquired upon the determined position of the local RF-coil and/or the known geometry of the local RF-coil. As mentioned, because of the known position of the coil, the detection of the localizer images can be restricted to the extension and/or the geometry of the RF-coil. This means that the localizer images are acquired over the region of interest and also that the regions next to the region of interest can be neglected. This has the advantage that the localizer images can be acquired with a high image quality, in particular during the initial patient table movement into the isocenter, without losing time.

According to a further embodiment of the invention, the localizer data are acquired along the x-direction perpendicular to the z-direction, and perpendicular to the longitudinal extent of the at least one local RF-coil. As also mentioned, because of the known position of the coil, the detection of the localizer images is restricted to the extent and/or the geometry of the RF-coil. This means that the localizer images are acquired solely in the region of interest and the regions next to the region of interest are neglected. This has the advantage that the localizer images can be acquired with a high image quality, in particular during the patient table is moved into the isocenter, without losing time.

In a further embodiment of the invention, the localizer data are acquired by execution of a computer program according to the known DICOM (Digital Imaging and

Communication in Medicine) standard, in particular the program FastView/TimCT. The localizer data are visually shown to an operator as soon as they are acquired, so that the operator, if necessary, can create a robust localizer that will include all relative anatomies, i.e. regions of interest, from patient-to-patient, or the operator can terminate the localizer when enough data have been obtained to continue with the study thereby minimizing extra effort. The FastView/TimCT is a 3D imaging acquisition, so all orientations are displayed. This enables the operator to quickly begin diagnostic imaging as the operator need not perform several 2D localizers. By using the DICOM standard, like FastView/TimCT, the diagnostic examination can be planned easily.

According to a further embodiment of the invention, the localizer data are reproduced as a localizer image or localizer images, in which at least one landmark is detectable, so that the patient table is re-adjustable, if necessary, such that a special region of interest is in the isocenter, even if the local RF-coil is not positioned exactly over the region of interest. The localizer data are visually presented to the operator so that the landmark can be detected easily within the localizer image. If an adjustment of the patient table is necessary, such as if the region of interest is not exactly in the isocenter along the z-direction, the patient table can be moved there easily, even if the local RF-coil is not exactly positioned on the region of interest. Such off-center imaging techniques are required if the region of interest is a region of a hip, or a region of an arm, or a region of a shoulder, or a region of a foot of a patient, or the like.

According to a further embodiment of the invention, the distance between the position of the at least one local RF-coil and the isocenter is determined by a camera system and/or a sensor system, in particular, by a sensor positioned inside the local RF-coil. If a camera system is used, it is preferably an optical camera system taking 2D or 3D images. In such an optical 2D or 3D image, a landmark can be easily identified. In case of a sensor which is alternatively or additionally used the sensor is preferably arranged inside or close to the RF-coil. Preferably, a Hall sensor is used which is a transducer that varies its output voltage dependent on a magnetic field. Thus, the RF coil is detected visually or by a Hall sensor and, because the dimensions of the RF coil are known, the localizer, i.e. the localizer data, needs to be scanned only from where the RF coil starts and ends.

The present invention also encompasses a medical imaging apparatus, specifically a magnetic resonance apparatus having a scanner with a basic field magnet and a patient table for placing a patient thereon, and at least one local RF-coil, which is positionable on or close to the patient on a region of interest which is to be examined. The apparatus also has a detector that detects distance between the position of the at least one local RF-coil and the isocenter of the scanner.

The apparatus has a control computer configured to automatically move the local RF-coil together with the patient table along the z-direction of the scanner for at least the determined distance, so that the center of the local RF-coil is in or at least at the z-position of the isocenter.

The inventive magnetic resonance imaging apparatus has a patient table onto which a patient is placed for an examination. The patient table is designed to be movable along the z-direction, in particular along a body length of a patient, from an exterior of the scanner of the magnetic resonance imaging apparatus into an interior of the scanner of the magnetic resonance imaging apparatus. Furthermore, at least one local RF-coil for receiving diagnostic data positionable on or close to the patient directly on a region of interest that is to be examined. For example, the local RF-coil may be plugged into a given connection socket at a predetermined location at the scanner. The distance between the position of the at least one local RF-coil and the isocenter of the basic field magnet, if the position is a fixed known point in the scanner, is determined automatically by the detection unit. However, the detection unit can be operated, if the position of the local RF-coil is not a predetermined position, to detect the position and the distance can then be calculated, such as the local RF coil is not directly plugged into a given connection. The detection unit may have the predetermined positions stored therein or may be operable to actively detect the position of the local RF-coil. The determination of the distance between the local RF-coil and the isocenter allows for movement of the local RF-coil together the patient table into the isocenter, such that the center of the local RF-coil is in or at least at the z-position of the isocenter. An advantage of the inventive magnetic resonance imaging apparatus is that it is configured to move the region of interest directly into the isocenter of the scanner. Using a laser pointer for adjustment is not needed anymore. This means the utilization of the magnetic resonance imaging apparatus can be increased.

According to an embodiment of the inventive magnetic resonance imaging apparatus, the magnetic resonance imaging apparatus has at least one predetermined position into which the at least one local RF-coil is pluggable such that the position of the least one local RF-coil is predetermined automatically. When the RF-coil is plugged into one of the predetermined positions, control computer of the magnetic resonance imaging apparatus directly and automatically detects which position is occupied. Since the pluggable positions and the isocenter are respectively fixed positions, the distance is stored as a function of the position of the patient table in the scanner in a control computer of the magnetic resonance imaging apparatus. The advantage of the predetermined positions is that the RF-coil used for a particular examination can be moved quickly into the isocenter without needing to determine the distance that the patient table needs to be moved.

According to an embodiment of the inventive magnetic resonance imaging apparatus, the magnetic resonance imaging apparatus has at least one detection unit that detects the least one local RF-coil. The at least one detection unit may be a camera system and/or a sensor located inside the at least one local RF-coil. Preferably, the detection unit is designed as a camera system which detects optical images. The optical images are either 2D or 3D images from which the distance between the RF-coil and the isocenter can be reliably calculated. Alternatively or additionally, the detection unit may be a sensor. Preferably, such sensor is arranged inside the RF-coil and is configured to measure an output voltage in response to a magnetic field, i.e., is configured as a Hall sensor. The advantage of the detection unit is that the RF-coil can be detected automatically even if the RF-coil is not arranged in a predetermined position.

According to another embodiment of the inventive magnetic resonance imaging apparatus, the magnetic resonance imaging apparatus is configured to acquire localizer data in the x-direction along the at least one local RF-coil, the x direction being perpendicular to the z-direction. Preferably, the localizer data are acquired along the longitudinal extent of the RF-coil, which is sufficient since the RF-coil is positioned over the region of interest to be examined. Therefore, it is sufficient to acquire a localizer image that primary reproduces the region of interest mainly in order to plan the diagnostic acquisition of imaging data in detail.

In another embodiment of the inventive magnetic resonance imaging apparatus, the magnetic resonance imaging apparatus is configured to acquire the localizer data at and/or near to the determined position of the local RF-coil and/or the known geometry of the local RF-coil. Because of the predetermined position of the local RF-coil and/or the known geometry of the local RF-coil, the localizer images can directly be acquired over the region of interest.

In an embodiment of the inventive magnetic resonance imaging apparatus, a computer program is executable by the control computer of the magnetic resonance imaging apparatus that uses the known DICOM (Digital Imaging and Communication in Medicine) standard, known as FastView/TimCT. This has the advantage that the proposed magnetic resonance imaging apparatus complies with such a standard.

The present invention also encompasses a non-transitory, computer-readable data storage medium encoded with programming instructions that, when the storage medium is loaded into a computer or computer system of medical imaging apparatus, such as magnetic resonance apparatus, cause the computer or computer system to operate the medical imaging apparatus in order to implement any or all of the embodiments of the method according to the invention, as described above.

### BRIEF DESCRIPTION OF THE DRAWINGS

FIG. 1 schematically illustrates a magnetic resonance imaging apparatus constructed and operating in accordance with the present invention.

FIG. 2 shows a patient placed on a patient table that is moved into a magnetic resonance modality.

FIG. 3 shows the patient placed on the patient table according to FIG. 2, wherein a RF-coil used for the particular examination has reached the isocenter of the magnetic resonance modality.

FIG. 4 is a flowchart of the inventive method.

FIG. 5 shows a center of a local RF-coil placed in the isocenter of the scanner of the magnetic resonance imaging apparatus

FIG. 6 shows localizer data acquired along the x-direction along the extent of the local RF-coil.

FIG. 7 shows a local RF-coil placed in the isocenter of the scanner of the magnetic resonance imaging apparatus, but wherein the center of the local RF-coil is not positioned in the isocenter.

### DESCRIPTION OF THE PREFERRED EMBODIMENTS

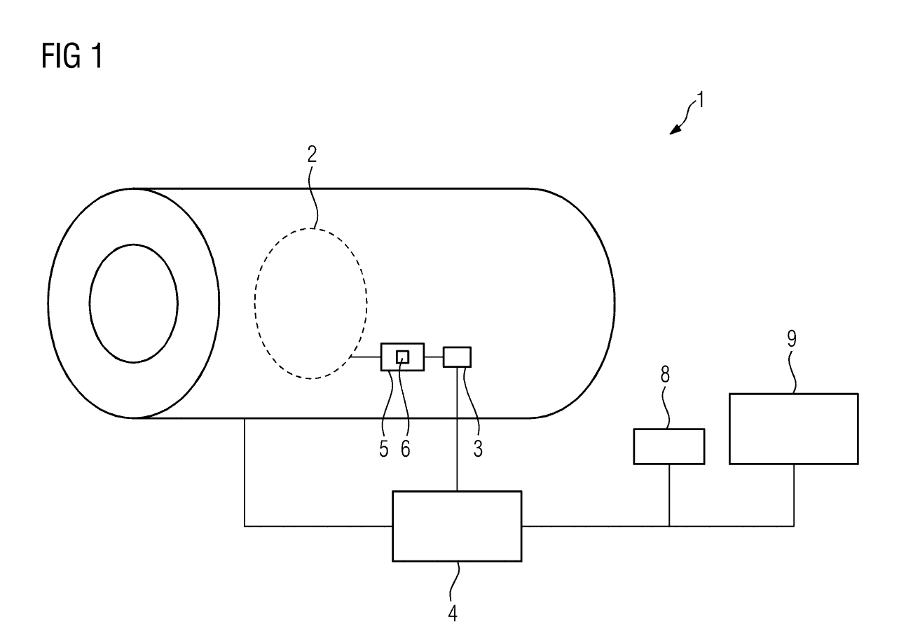

The present invention is explained in the following with respect to FIGS. 1 to 7.

FIG. 1 shows a scanner of a magnetic resonance imaging apparatus, in particular a magnetic resonance apparatus, having at least one RF-coil 2. The RF-coil 2 is connected to a control computer 4 of the magnetic resonance imaging apparatus through an electronic connection 3. The connection 3 can be configured as a plug-in connector. The connection 3 allows for connection of different RF-coils with the control computer 4. The illustration of the magnetic resonance imaging apparatus according to FIG. 1 is simplified. Several components, i.e., more than only the connection 3, are usually arranged between the RF-coil 2 and the control computer 4. The connection 3 allows for changing between different RF-coils 2.

The RF-coil 2 has an information element 5, including an information code 6. The information code 6 provides a coil identification. For example, the identifications code 6 can be a series of numbers such as 124, which identify an RF-coil used in the examination of hips.

Furthermore, a display unit 8 and an input unit 9 are both connected with the control computer 4. The display unit 8 displays, for example, the localizer data in the form of localizer images or the like.

The method disclosed herein is preferably implemented as a computer program, i.e., software, in the control computer 4.

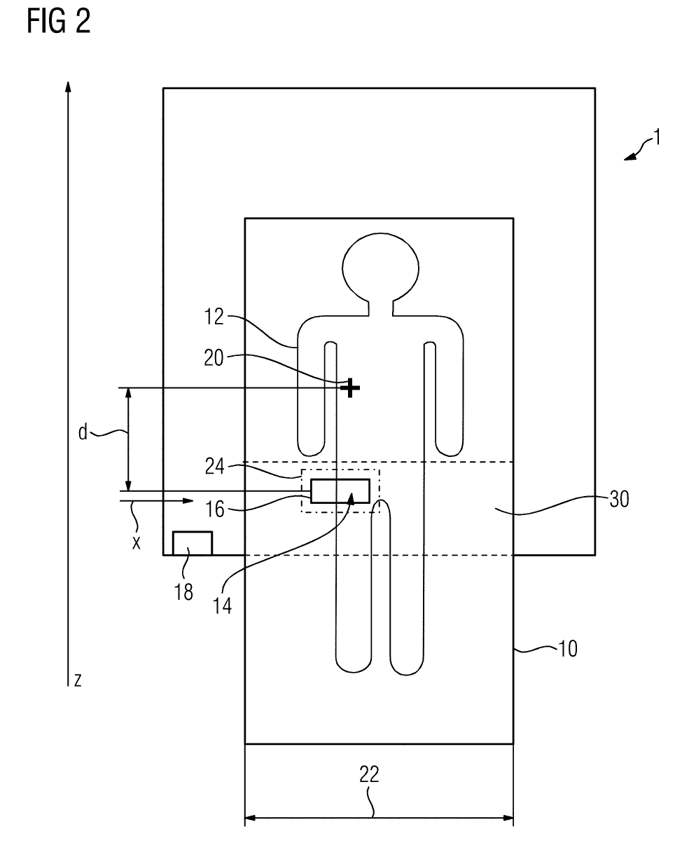

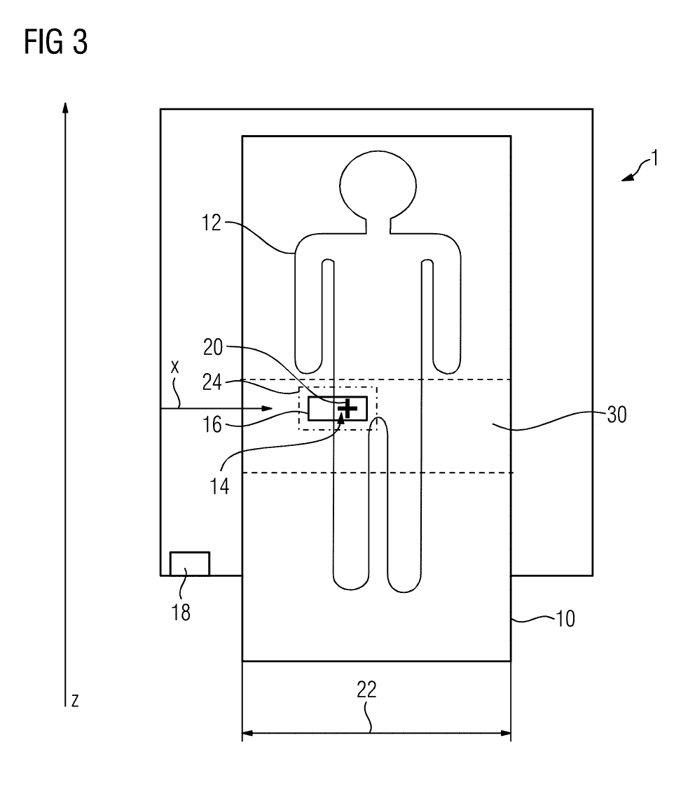

FIGS. 2 and 3 each show a schematical top view of the magnetic resonance imaging apparatus 1. The magnetic resonance imaging apparatus has a patient table 10 movable along the z-direction (compare with the patient table position of FIGS. 2 and 3). A patient 12 is placed on the patient table 10, whose hip 14 is to be examined by the scanner 1.

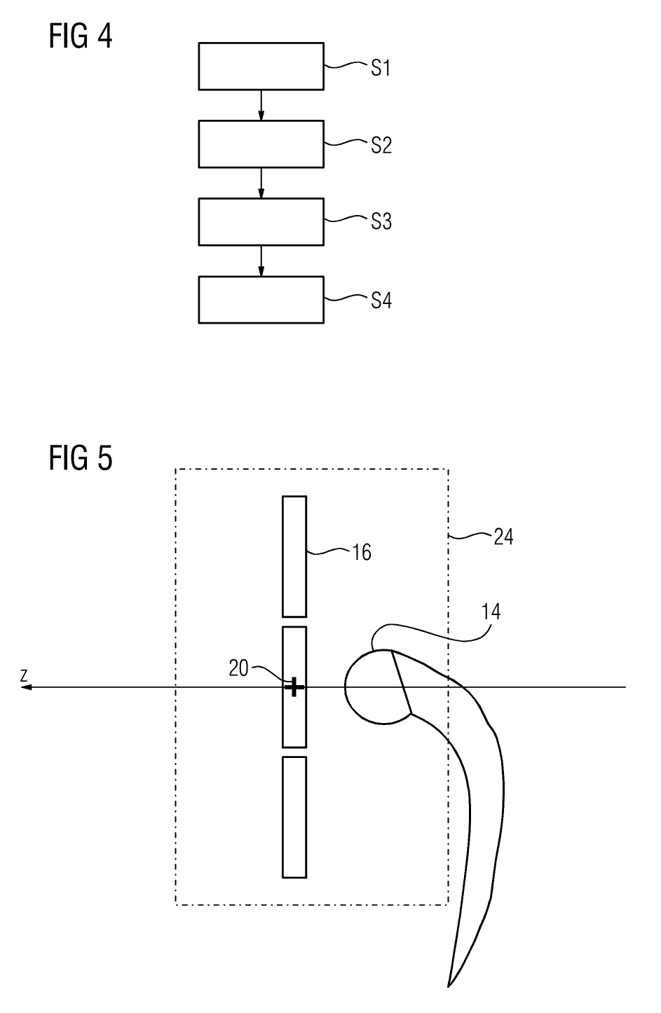

In order to perform a diagnostic examination, for example, of a patient's hip 14, the patient 12 is placed on the patient table 10 in a first step 51 as shown in FIG. 4. Thereafter a local RF-coil 16 is placed on or close over a region of interest, for example, a region of the hip 14 (compare with FIG. 2 or 3). If the local RF-coil 16 is plugged into a predetermined position, the magnetic resonance imaging apparatus will automatically detect the position of the local RF-coil 16. Alternatively or additionally, a detection unit 18 detects the position of the local RF-coil 16. The detection unit 18 supplies the detected position of the local RF-coil 16 to the control computer 4.

In a second step S2, the control computer 4 evaluates a distance d between the position of the local RF-coil 16 and an isocenter 20 of the scanner 1. After determination of the distance d according to a third step S3 indicated in FIG. 4, the patient table 10 is moved the distance d along the z-direction such that the local RF-coil 16 is in or at least at or near to the z-position of the isocenter 20 (compare with FIGS. 2 and 3). FIG. 5 shows the local RF-coil 16, and the center of the local RF-coil 16 is placed in the isocenter 20 of the basic field magnet.

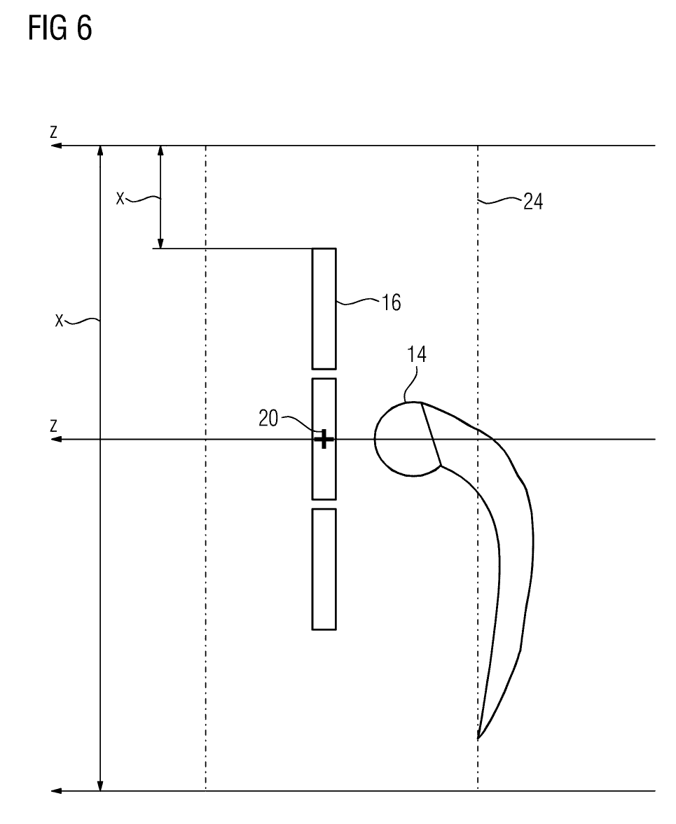

As the patient table 10 is moved the distance d along the z-direction, localizer data are acquired along an x-direction, which is perpendicular to the z-direction. Preferably, the x-direction extends in parallel to a width 22 of the patient table 10. In particular, the localizer imaging is started when the patient table 10 is moved along the z-direction. Because of the determined position of the local RF-coil 16 and/or its known geometry, the localizer data acquisition starts at a position x before the local RF-coil 16 and ends a position x behind the local RF-coil 16, such that the localizer data are preferably acquired in the region of interest 24 (compare with FIG. 6). It is also possible to stop the localizer data acquisition at a point x that is located above the local RF-coil 16 or close behind the local RF-coil 16 (not shown). However, the localizer data are acquired along an x-direction and along a z-direction, so that the localizer data comprises the region of interest 24. Because a patient's weight, height, or the like are input into the magnetic resonance imaging apparatus 1, it is not necessary to detect localizer data along the y-direction being perpendicular to the z- and the x-directions. If, for example, a patient 12 is overweight, the control computer 4 of the magnetic resonance imaging apparatus is configured to reliably estimate where in y-direction the examination should be focused. Such acquisition of localizer data could be performed using, for example, a body coil or with a surface coil, which geometry and in particular which extension are known. An advantage is that no prescan data are needed to begin the localization work step. In particular, the imaging of localizer data, i.e., localizer images, is performed using a computer program, such as FastView/TimCT.

Preferably, the localizer image or the localizer images will be acquired until a position x after the local RF-coil 16 or the local RF-coils 16 to obtain a good overview. It is also possible that more than one local RF-coil 16 is positioned in the z-direction and/or x-direction (not shown). Since the information corresponding to a region 30 of a left and a right position of the local RF-coil 16 is usually taken into account, it is possible to reduce the localizer data acquired to the region of interest 24, i.e., to one side. In case of a hip examination, the one side corresponds to the hip which is to be examined. In particular, such a procedure is useful for off-center imaging techniques, for example if a hip, an arm, a foot, a shoulder or the like shall be examined.

After the acquisition of localizer data, in particular in the region of interest 24, the patient table 10 is moved back into the isocenter 20, or at least to the z-coordinate of the isocenter 20.

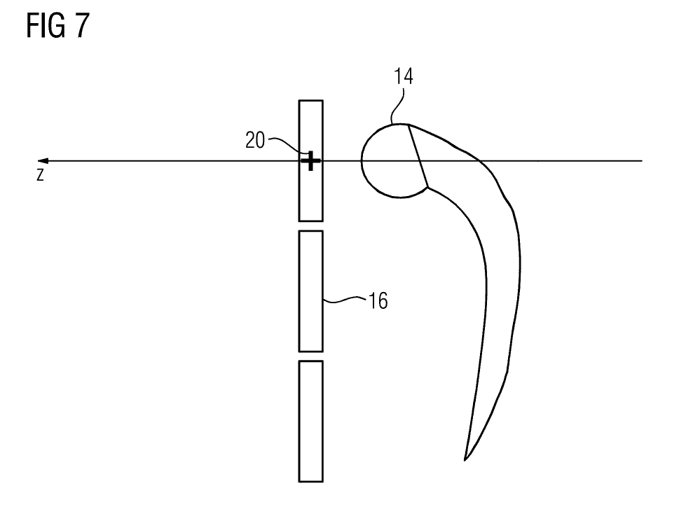

After the image acquisition, landmarks can be detected inside the image and the patient table 10 can be moved along the z-direction such that a particular anatomy of the patient is in the isocenter which refers to an optional work step S4 (compare with FIG. 4). According to the example of the hip examination, the hip 14 can be detected and the patient table 10 is moved in such a way that the femoral head is in the isocenter 20, even when the local RF-coil 16, the center of the local RF-coil 16, is not positioned exactly over the hip (compare with FIG. 7).

Thus, a particularly preferred inventive method can be summarized as follows:
1. The patient 12 is placed on the patient table 10.
2. The RF coil 16 is placed over the region of interest 24.
3. The RF coil 16 position is automatically detected via a camera and/or a Hall sensor detection.
4. When the operator selects a “move to center icon”, the patient table 10 begins to move to the isocenter along the z-direction of the patient table 10.
5. As the RF coil 16 that is detected by the system approaches the isocenter, the FastView/TimCT begins automatically to scan until the end of the detected RF-coil in the z direction and/or x-direction. This allows for a more generous size localizer to minimize missed anatomy in the localizer. This is done in one patient table 10 movement.
6. If the camera or the Hall sensor detects a “non isocenter” position of the coil in x- or y-direction, the small localizer can then be shifted to the appropriate location as detected. The localizer will still scan in z-direction as the patient table 10 moves to the isocenter just with a shift in x and y axis to the region of interest 24.

Advantages of the disclosed method are
no localizer light is needed
one button to diagnostic imaging capabilities is needed
less experienced personnel can perform patient setup.

Although modifications and changes may be suggested by those skilled in the art, it is the intention of the Applicant to embody within the patent warranted hereon all changes and modifications as reasonably and properly come within the scope of the Applicant's contribution to the art.

1. A method for direct positioning of a region of interest of a patient inside a basic field magnet of a magnetic resonance (MR) data acquisition scanner of an MR imaging apparatus, said basic field magnet having an isocenter, said method comprising:
positioning a patient on a patient table that is movable with respect to said MR data acquisition scanner;
positioning at least one local radio-frequency (RF) coil on or adjacent to the patient on a region of interest of the patient, from which MR data are to be acquired;
in a computer of the MR imaging apparatus, determining a distance between a position of said at least one local RF coil and the isocenter of the basic field magnet; and
automatically controlling movement of said patient table, with said patient and said at least one local RF coil thereon, along a z-direction that proceeds through said basic field magnet, through at least said predetermined distance in order to move a center of said at least one local RF coil into said isocenter or at least to a position of the isocenter along said z-direction.

2. A method as claimed in claim 1 comprising operating a detection unit to detect the position of said at least one local RF coil, and providing a position signal from said detection unit to said computer representing said position of said at least one local RF coil.

3. A method as claimed in claim 1 comprising, from said computer, operating said MR data acquisition scanner to acquire localizer data of the patient while said patient table, with said patient and said at least one local RF coil thereon, is moved along said z-direction.

4. A method as claimed in claim 3 comprising acquiring said localizer data so as to encompass said position of said at least one local RF coil.

5. A method as claimed in claim 4 comprising also acquiring said localizer data along a x-direction, that is perpendicular to the z-direction, and perpendicular to an extent of said at least one local RF coil along said z-direction.

6. A method as claimed in claim 3 comprising acquiring said localizer data by executing a localizer data acquisition algorithm that operates according to the DICOM (Digital Imaging and Communication in Medicine) standard.

7. A method as claimed in claim 3 comprising, in said computer, reconstructing said localizer data into localizer image data in which at least one landmark is detectable that is associated with at least one of the patient or the at least one local RF coil, and using said landmark to ensure that said region of interest in said isocenter, even when said at least one local RF coil is not positioned exactly on the region of interest.

8. A method as claimed in claim 1 comprising placing said at least one local RF coil on a region of interest of the patient selected from the group consisting of a region of a hip, a region of an arm, a region of a shoulder, and a region of a foot.

9. A method as claimed in claim 1 comprising determining said distance between the position of said at least one local coil and said isocenter by operating a detector system, selected from the group consisting of a camera system and a sensor system, in order to detect said position of said at least one local RF coil.

10. A magnetic resonance (MR) imaging apparatus comprising:
an MR data acquisition scanner comprising a basic field magnet having an isocenter;
a patient table, adapted to receive a patient thereon, that is movable relative to said MR data acquisition scanner;
at least one local radio-frequency (RF) coil that is selectively placeable on or adjacent to the patient on a region of interest of the patient, from which MR data are to be acquired;
a computer configured to determine a distance between a position of said at least one local RF coil and the isocenter of the basic field magnet; and
said computer being configured to automatically control movement of said patient table, with said patient and said at least one local RF coil thereon, along a z-direction that proceeds through said basic field magnet, through at least said predetermined distance in order to move a center of said at least one local RF coil into said isocenter or at least to a position of the isocenter along said z-direction.

11. An MR imaging apparatus as claimed in claim 10 wherein said at least one local RF coil comprises a plug and wherein said MR data acquisition scanner comprises a socket, at a predetermined position in said MR data acquisition scanner, that receives said plug therein, and wherein said computer is configured to determine the position of said at least one local RF coil automatically from said predetermined position of said socket.

12. An MR imaging apparatus as claimed in claim 10 comprising a detection unit that detects the position of said at least one local RF coil.

13. An MR imaging apparatus as claimed in claim 12 wherein said detection unit is selected from the group consisting of a camera and a sensor.

14. MR imaging apparatus as claimed in claim 10 wherein said computer is configured to operate the MR data acquisition scanner in order to acquire localizer data in an x-direction, that is perpendicular to said z-direction, along an extent of said at least one local RF coil.

15. MR imaging apparatus as claimed in claim 13 wherein said computer is configured to operate the MR data acquisition scanner in order to acquire said localizer data from a position in said MR data acquisition scanner at or near said position of said at least one local RF coil.

16. MR imaging apparatus as claimed in claim 10 wherein said computer is configured to operate the MR data acquisition scanner in order to acquire said localizer data according to the DICOM (Digital Imaging and Communication in Medicine) standard.

17. A non-transitory, computer-readable data storage medium encoded with programming instructions, said storage medium being loaded into a computer or computer system of a magnetic resonance (MR) imaging apparatus comprising an MR data acquisition scanner having a basic field magnet with an isocenter, a patient table, adapted to receive a patient thereon that is movable relative to the MR data acquisition scanner, and at least one local RF coil that is selectively placeable on or adjacent to the patient on a region of interest of the patient, from which MR data are to be acquired, said programming instructions causing said computer or computer system to operate the MR imaging apparatus to:
determine a distance between a position of said at least one local RF coil and the isocenter of the basic field magnet; and
automatically control movement of said patient table, with said patient and said at least one local RF coil thereon, along a z-direction that proceeds through said basic field magnet, through at least said predetermined distance in order to move a center of said at least one local RF coil into said isocenter or at least to a position of the isocenter along said z-direction.
